(19) United States
(12) Patent Application Publication
(10) Pub. No.: US 2008/0228128 A1
(43) Pub. Date: Sep. 18, 2008

(54) Applicator having an intermittent gripping structure

(76) Inventors: Nancy Karapasha, Cincinnati, OH (US); Dean Jeffery Daniels, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/724,117

(22) Filed: Mar. 14, 2007

(51) Int. Cl. A61F 13/32

(52) U.S. Cl. 604/15

(57) ABSTRACT

An applicator having an intermittent gripping structure. The applicator can have an insertion member having an insertion end and a gripping portion opposite the insertion end. The gripping portion can have at least one substantially continuous ring disposed along substantially the entire circumference of the gripping portion and can have two or more elongated ridges extending along at least a portion of the circumference. The two or more elongated ridges can have a length extending perpendicular to a longitudinal axis of the applicator and a width, and the length can be greater than the width. The two or more ridges can extend along less than substantially the entire circumference of the gripping portion.

### FIELD OF THE INVENTION

The present invention relates generally to applicators having an intermittent gripping structure, more specifically to applicators for feminine care products having an intermittent gripping structure.

### BACKGROUND OF THE INVENTION

Feminine care products, such as tampons and pessaries, are generally used by women within the vagina for feminine needs, such as, e.g., to absorb menstrual or other body exudates, for pelvic support, and/or for other feminine needs. Such feminine products can be inserted into the vagina digitally, such as, e.g., by using a finger, or can be inserted into the vagina by using an applicator.

Applicators typically can comprise an insertion member and a plunger. The material to be expelled from the applicator, such as an absorbent tampon or pessary, can be positioned within the insertion member. The insertion member can have a first end for insertion of the material and a second end for receipt of the plunger. To use the applicator, the consumer will grasp the insertion member, position the first end appropriately, such as, e.g., into the body, and move the plunger in the insertion member towards the first end to insert the material. Some applicators can also include a fingergrip configuration that is located on the insertion member, which can allow the consumer to more securely hold the applicator during insertion of a material into the body cavity. Various fingergrip configurations have been utilized to facilitate the handling of the applicator and to improve the insertion experience. Currently available configurations, however, are not yet optimized to consistently deliver such benefits.

As such, it would be desirable to provide an applicator having improved ease of insertion. It would also be desirable to provide an applicator having an enhanced gripping structure.

### SUMMARY OF THE INVENTION

An applicator having an intermittent gripping structure is provided. The applicator can include an insertion member having an insertion end and a gripping portion opposite the insertion end. The gripping portion can have at least one substantially continuous ring disposed along substantially the entire perimeter, such as, e.g., substantially the entire circumference, of the gripping portion and can have two or more elongated ridges extending along at least a portion of the perimeter. The two or more elongated ridges can have a length extending perpendicular to a longitudinal axis of the applicator and a width, and the length can be greater than the width.

### BRIEF DESCRIPTION OF THE DRAWINGS

FIG. 1 is a side view of an applicator of the present invention.

FIG. 2 is a view showing the entire perimeter of a gripping portion of the present invention.

FIG. 3 is a side of an applicator of the present invention.

FIG. 4 is a side view of an applicator of the present invention.

FIG. 5 is a side view of an applicator of the present invention.

FIG. 6 is a side view of an applicator of the present invention.

FIG. 7 is a view showing the entire perimeter of a gripping portion of the present invention.

FIG. 8 is a view showing the entire perimeter of a gripping portion of the present invention.

FIG. 9 is a side view of a plunger of the present invention.

FIG. 10 is a side view of an applicator of the present invention.

### DETAILED DESCRIPTION OF THE INVENTION

Applicators having an intermittent gripping structure are provided. Such an enhanced gripping structure can provide for improved ease of insertion, enhanced grippability, and/or improved tube lock. In certain embodiments, an intermittent gripping structure can be arranged to provide a finger landing area, such as, e.g., to improve grippability and/or control prior to and during insertion.

As used herein, the term “feminine care product” includes absorbent articles useful for feminine needs, such as articles that typically can be intended for feminine use internally, such as, e.g., within a user's vagina. Internal feminine care products can include, for example, tampons and pessaries.

As used herein, the term “tampon” refers to any type of absorbent structure that can be inserted into the vaginal canal or other body cavity, such as, e.g., for the absorption of fluid, to aid in wound healing, and/or for the delivery of materials, such as moisture or active materials such as medicaments.

As used herein, the term “pessary” refers to any type of substantially non-absorbent structure for the purpose of reducing urine leakage and/or supporting a prolapsed uterus and/or bladder. Such pessaries can have any variety of shapes and sizes including cylinder, ovate, spherical, tubular, annual rings, “U” shaped, cup shaped, rings, cubes or donut shaped, and can function in any suitable manner, such as, e.g., by direct application of support, lever force, expansion of the device by selection of material, and/or by inflation of the device.

As used herein, the term “vaginal canal” refers to the internal genitalia of the human female in the pudendal region of the body. The terms “vaginal canal” or “within the vagina” as used herein are intended to refer to the space located between the introitus of the vagina (sometimes referred to as the sphincter of the vagina) and the cervix.

As used herein, “applicator” refers to a device or implement that facilitates the insertion of a feminine care product, such as, e.g., a tampon or pessary, into an external orifice of a mammal. Exemplary applicators include telescoping, tube and plunger, and compact applicators.

As used herein, the term “insertion end” refers to the portion of the tampon or applicator including the end that is intended to enter the vaginal canal first when inserting the tampon or applicator into the vaginal canal.

As used herein, the term “withdrawal end” refers to the portion of the applicator opposite the insertion end including the end is intended to exit the vaginal canal first when the applicator is removed from the vagina.

As used herein, the term “gripping formations” refers to raised structures provided at the gripping portion of the applicator to assist a user in grasping the applicator. Suitable gripping formations include, e.g., projections, rings, ridges, ribs, embossments, and/or other raised surfaces.

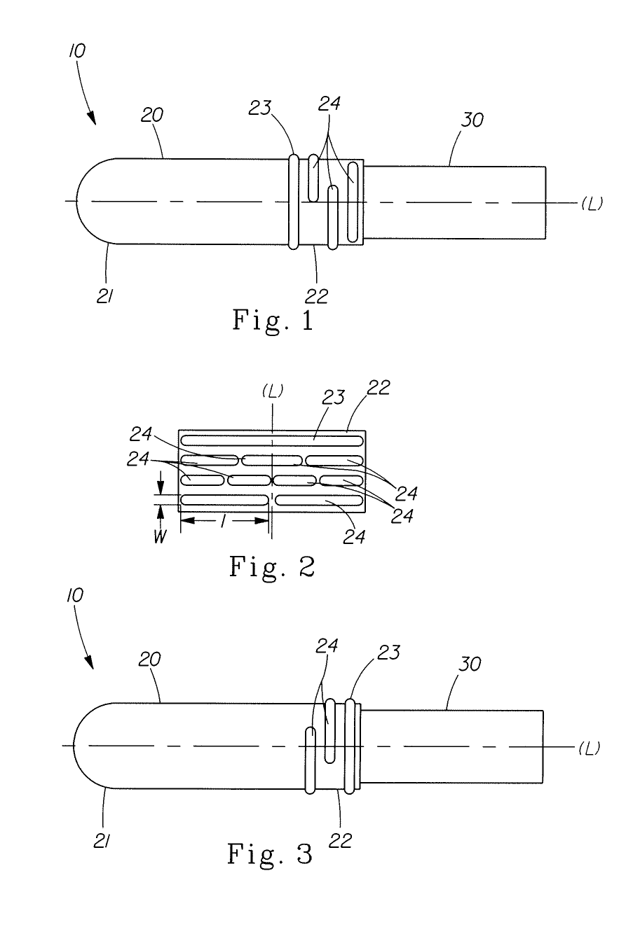

FIG. 1 shows one embodiment of an applicator 10. The applicator 10 comprises an insertion member 20 and a plunger 30. The insertion member 20 has an insertion end 21 and a gripping portion 22 opposite the insertion end 21. The gripping portion 22 can have a series of gripping formations, such as, e.g., at least one substantially continuous ring 23 disposed along substantially the entire perimeter, such as, e.g., substantially the entire circumference, of the gripping portion 22 in a direction generally perpendicular to the longitudinal axis (L) of the applicator 10. The gripping portion 22 can also have two or more elongated ridges 24 that can be disposed along at least a portion of the perimeter of the gripping portion 22 but that do not extend along substantially the entire perimeter of the gripping portion 22. As shown in FIG. 1, in certain embodiments, the gripping portion 22 can include a substantially continuous ring 23 followed by three elongated ridges 24, each disposed at a different distance along the longitudinal axis (L) of the applicator 10, with the substantially continuous ring 23 provided in the gripping portion at a location along the longitudinal axis (L) closest to the insertion end of the applicator 10 compared to the location of the elongated ridges 24. The elongated ridges 24 can be the same size or different sizes. As shown in FIG. 1, the elongated ridges 24 can be arcuate, such as, e.g., in embodiments where the gripping portion 22 has a generally circular, oval, elliptical, or other suitable cross-section. Alternatively, however, the elongated ridges 24 need not be arcuate, such as, e.g., in embodiments where the gripping portion 22 has a cross-section having a non-arcuate perimeter, such as, e.g., a square, rectangular, triangular, polygonal, flattened, or other suitable cross-sectional shape. In certain embodiments, the gripping portion 22 can have a perimeter wherein a portion of the perimeter is arcuate and wherein a portion of the perimeter is non-arcuate, such as, e.g., a gripping portion with one or more curved sides and one or more flattened sides.

FIG. 2 shows view of substantially the entire circumferential area of a gripping portion 22 of one embodiment of the invention. As shown in FIG. 2, each ridge 24 can have a length (l) perpendicular to the longitudinal axis (L) of the applicator 10 and a width (w), with the length (1) of the ridge 24 being longer than the width (w). In certain embodiments, each elongated ridge 24 can be disposed at a different distance from the insertion end 21 along the longitudinal axis (L) of the applicator 10. In addition, or alternatively, one or more elongated ridges 24 can be disposed at a same distance from the insertion end 21 along the longitudinal axis (L) of the applicator 10.

FIG. 3 shows one embodiment of an applicator 10. The applicator 10 comprises an insertion member 20 and a plunger 30. The insertion member 20 has an insertion end 21 and a gripping portion 22 opposite the insertion end 21. The gripping portion 22 can have a series of gripping formations, such as, e.g., at least one substantially continuous ring 23 disposed along substantially the entire perimeter, such as, e.g., substantially the entire circumference, of the gripping portion 22 in a direction generally perpendicular to the longitudinal axis (L) of the applicator 10. As shown in FIG. 3, the gripping portion 22 can also have two or more elongated ridges 24 that can be disposed along at least a portion of the perimeter of the gripping portion 22 but that do not extend along substantially the entire perimeter of the gripping portion 22. In this embodiment, the gripping portion 22 can include two elongated ridges 24 followed by a substantially continuous ring, each disposed at a different distance along the longitudinal axis (L) of the applicator 10, with the substantially continuous ring 23 provided in the gripping portion at a location along the longitudinal axis (L) closest to the withdrawal end of the applicator 10 compared to the location of the elongated ridges 24. In certain embodiments, the gripping portion 22 can have three or more, four or more, five or more, six or more, seven or more, eight or more, nine or more, ten or more, or any other suitable number, of ridges 24.

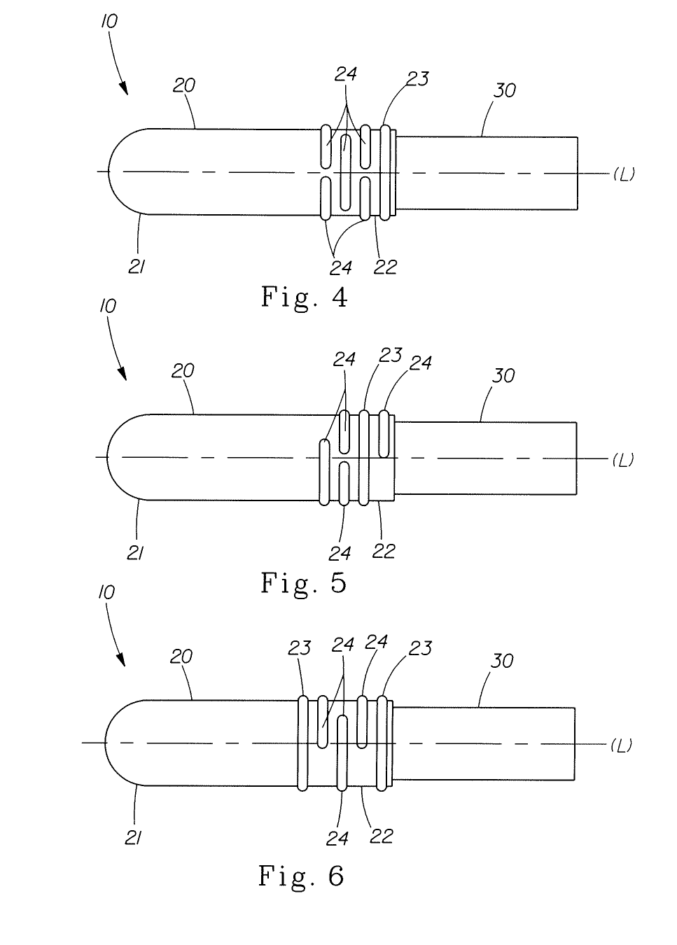

FIG. 4 shows one embodiment of an applicator 10. The applicator 10 comprises an insertion member 20 and a plunger 30. The insertion member 20 has an insertion end 21 and a gripping portion 22 opposite the insertion end 21. In this embodiment, the gripping portion 22 has a series of gripping formations that can include a substantially continuous ring 23 disposed along substantially the entire perimeter, such as, e.g., substantially the entire circumference, of the gripping portion 22 at a first distance from the insertion end 21 along the longitudinal axis (L) of the applicator 10, two elongated ridges 24 disposed at a second distance from the insertion end 21 along the longitudinal axis (L) of the applicator 10, a elongated ridge 24 disposed at a third distance from the insertion end 21 along the longitudinal axis (L) of the applicator 10, and two additional elongated ridges 24 disposed at a fourth distance from the insertion end 21 along the longitudinal axis (L) of the applicator 10. As shown in FIG. 4, the first distance can be a distance closest to the withdrawal end of the applicator 10. Alternatively, the first distance can be a distance closest to the insertion end 25 of the applicator 10 or at any other suitable distance along the gripping portion.

FIG. 5 shows one embodiment of an applicator 10. The applicator 10 comprises an insertion member 20 and a plunger 30. The insertion member 20 has an insertion end 21 and a gripping portion 22 opposite the insertion end 21. In this embodiment, the gripping portion 22 has a series of gripping formations that can include a substantially continuous ring 23 disposed along substantially the entire perimeter, such as, e.g., the entire circumference of the gripping portion 22 at a first distance from the insertion end 21 along the longitudinal axis (L) of the applicator 10, an elongated ridge 24 disposed at a second distance from the insertion end 21 along the longitudinal axis (L) of the applicator 10, two elongated ridges 24 disposed at a third distance from the insertion end 21 along the longitudinal axis (L) of the applicator 10, and an additional elongated ridge 24 disposed at a fourth distance from the insertion end 21 along the longitudinal axis (L) of the applicator 10. In certain embodiments, any suitable number of additional ridges can be disposed on the gripping portion 22, such as, e.g., additional ridges disposed at the second distance, the third distance, the fourth distance, or any other suitable distance from the insertion end 21 along the longitudinal axis (L) of the applicator 10.

FIG. 6 shows one embodiment of an applicator 10. The applicator 10 comprises an insertion member 20 and a plunger 30. The insertion member 20 has an insertion end 21 and a gripping portion 22 opposite the insertion end 21. In this embodiment, the gripping portion 22 has a series of gripping formations that can include a substantially continuous ring 23 disposed along substantially the entire perimeter, such as, e.g., substantially the entire circumference, of the gripping portion 22 at a first distance from the insertion end 21 along the longitudinal axis (L) of the applicator 10, a elongated ridge 24 disposed at a second distance from the insertion end 21 along the longitudinal axis (L) of the applicator 10, a elongated ridge 24 disposed at a third distance from the insertion end 21 along the longitudinal axis (L) of the applicator 10, a elongated ridge 24 disposed at a fourth distance from the insertion end 21 along the longitudinal axis (L) of the applicator 10, and a substantially continuous ring 23 disposed along substantially the entire perimeter, such as, e.g., the entire circumference of the gripping portion 22 at a fifth distance from the insertion end 21 along the longitudinal axis (L) of the applicator 10. In certain embodiments, any suitable number of additional ridges can be disposed on the gripping portion 22, such as, e.g., additional ridges disposed at the second distance, the third distance, the fourth distance, or any other suitable distance from the insertion end 21 along the longitudinal axis (L) of the applicator 10.

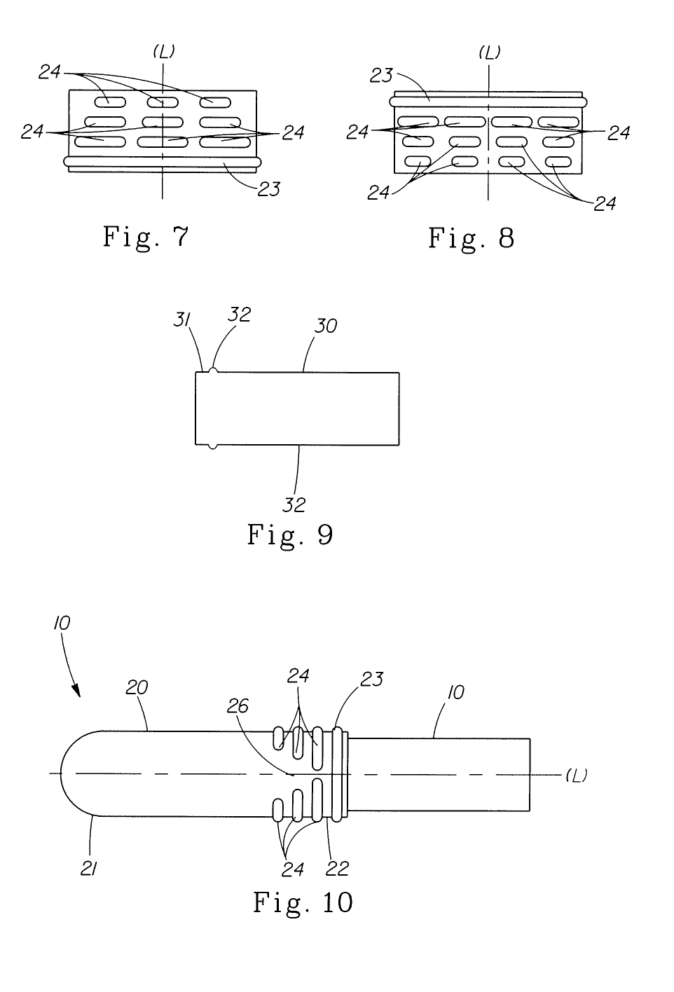

FIG. 7 shows a view of substantially the entire circumferential area of one embodiment of a gripping portion 22 of the present invention. As shown in FIG. 7, the gripping portion 22 can include a substantially continuous ring 23 and a series of elongated ridges 24. In this embodiment, the gripping portion 22 includes three elongated ridges 24 at a first distance from the insertion end along the longitudinal axis (L) of the applicator, three elongated ridges 24 at a second distance from the insertion end along the longitudinal axis (L) of the applicator, three elongated ridges 24 at a third distance from the insertion end along the longitudinal axis (L) of the applicator, and a substantially continuous ring 23 at a fourth distance from the insertion end along the longitudinal axis (L) of the applicator.

FIG. 8 shows a view of substantially the entire circumferential area of a gripping portion 22 of the present invention. As shown in FIG. 8, the gripping portion 22 can include a substantially continuous ring 23 and a series of elongated ridges 24. In this embodiment, the gripping portion 22 includes four elongated ridges 24 at a first distance from the insertion end along the longitudinal axis (L) of the applicator, four elongated ridges 24 at a second distance from the insertion end along the longitudinal axis (L) of the applicator, four elongated ridges 24 at a third distance from the insertion end along the longitudinal axis (L) of the applicator, and a substantially continuous ring 23 at a fourth distance from the insertion end along the longitudinal axis (L) of the applicator.

FIG. 9 shows one embodiment of a plunger 30. The plunger 30 can be telescopically and slidably mounted in the insertion member 20, such as, e.g., shown in FIGS. 1 and 3-6. The plunger 30 can have a first end 31 adapted for insertion into the insertion member 20 and a second end 32 opposite the first end 31. The first end 31 can have at least one raised projection 32 adapted to interact with one or more substantially continuous rings 23 on the gripping portion 22 of the insertion member 20, such as, e.g., the substantially continuous rings 23 shown in FIGS. 1-8. The raised portion 32 can be adapted to interact with a substantially continuous ring 23 of the insertion member 20 such that inadvertent separation of the insertion member 20 and the plunger 30 can be reduced. In certain embodiments, the raised portion 32 can interlock with a substantially continuous ring 23 of the insertion member 20 to inhibit removal of the first end 31 of the plunger 30 from the insertion member 20 absent the application of mechanical force. In certain embodiments, the plunger can be an optional component for use with the applicator. For example, the applicator can be fully functional if the plunger is omitted, i.e., if a user must insert and push the feminine care product through the tampon applicator digitally.

FIG. 10 shows one embodiment of an applicator 10. The applicator 10 can include an insertion member 20 and a plunger 30. The insertion member 20 has an insertion end 21 and a gripping portion 22 opposite the insertion end 21. In this embodiment, the gripping portion 22 can have a series of gripping formations, such as, e.g., at least one substantially continuous ring 23 disposed along substantially the entire perimeter, such as, e.g., substantially the entire circumference, of the gripping portion 22 in a direction generally perpendicular to the longitudinal axis (L) of the applicator 10 and a series of elongated ridges 24 that can be disposed along at least a portion of the perimeter of the gripping portion 22 but that do not extend along substantially the entire perimeter of the gripping portion 22. As shown in FIG. 10, in certain embodiments, the gripping formations can be arranged to provide an area along the gripping portion lacking gripping formations, such as, e.g., a landing area 26 suitable for a user to place a finger or thumb.

The insertion member can be constructed from any suitable material. Suitable materials include, for example, paper, paperboard, cardboard, cellulose, such as, e.g., molded cellulose, or any combinations thereof, polyethylene, polypropylene, polybutylene, polystyrene, polyvinylchloride, polyacrylate, polymethacrylate, polyacrylonitrile, polyacrylamide, polyamide, nylon, polyimide, polyester, polycarbonate, polylactic acid, poly hydroxyalkanoate, ethylene vinyl acetate, polyurethane, silicone, derivatives thereof, copolymers thereof, mixtures thereof, or any suitable smooth plastic material. Examples of suitable materials are disclosed in, e.g., U.S. Pat. Nos. 5,346,468 and 5,558,631. In certain embodiments, additives can be included in the material to alter or enhance certain material properties. Suitable additives include, for example, mold release agents, slip agents, surface energy modifiers, pearlescent agents, and/or any other suitable additives. In certain embodiments, the insertion member can be coated with a substance to give it a high slip characteristic, such as, e.g., with wax, polyethylene, a combination of wax and polyethylene, cellophane, clay, mica, and other lubricants that can facilitate comfortable insertion. Alternatively, or in addition, the insertion member can include a textured surface. Texture can be provided in any suitable manner, such as, e.g., by designing texture into or adding texture to the insertion member.

In certain embodiments, the insertion member can be in the form of a spirally wound, convolutely wound or longitudinally seamed hollow tube, which can be formed from paper, paperboard, cardboard or a combination thereof. The insertion member can have one or more walls of any suitable thickness. In certain embodiments, the one or more walls can have a predetermined thickness of from about 0.1 millimeters to about 0.7 millimeter. The wall can be constructed from a single ply of material or can be formed from two or more plies that are bonded together, such as, e.g., to form a laminate. When two or more plies are utilized, some or all of the plies can be spirally wound, convolutely wound or longitudinally seamed to form an elongated cylinder. For example, in certain embodiments the wall can be constructed using a smooth thin ply of material on the outside or exterior surface that surrounds a coarser and possibly thicker ply. In embodiments where the wall contains at least three plies, the middle ply can be the thicker ply and the interior and exterior plies can be smooth and/or slippery to facilitate expulsion of the tampon and to facilitate insertion of the insertion member. The wall can contain one to four plies, although more plies can be utilized if desired.

The plies can be held together in any suitable manner, such as, e.g., by one or more adhesives, such as glue, by heat, by pressure, by ultrasonics, or by any other suitable manner for holding the plies together. The adhesive can be either water-soluble or water-insoluble. In certain embodiments, a water-soluble adhesive can be used such that the wall will quickly break apart when it is immersed in water, such as, e.g., by flushing the insertion member down a toilet.

Alternatively, the material can be overlapped into a tubular configuration, such as, for example, by spirally or convolutely winding the insertion member into a cylindrical tube. In the case of other tube construction methods such as fiber or plastic molding, or integral tube forming (e.g., thermoforming plastic) no seams may be present and the corrugations could optionally be formed as part of the tube molding or forming process.

As set forth herein, the insertion member includes a gripping portion having a plurality of gripping formations, such as, e.g., projections, rings, ridges, ribs, embossments, depressions, grooves, and/or other gripping structures. The gripping formations can be provided in any suitable manner, such as, e.g., by the addition of material, and/or by impressing, such as, e.g., by embossing, or compressing the surfaces. In certain embodiments, the grip portion can include one or more flattened sides and/or one or more spaces for a decorative marking or a character, such as, e.g., an embossed and/or printed marking or character. In addition, or alternatively, the surfaces of the grip portion can include a material that can provide a frictional resistance for the user's fingers during the insertion of the tampon applicator into the body. Suitable materials that can provide friction include, for example, abrasive materials, high wet coefficient of friction materials, pressure sensitive adhesives, or any combinations thereof.

The gripping formations can have any suitable dimensions. In certain embodiments, the gripping formations can be elongated, such as, e.g., having a length greater than the width. In addition or alternatively, the gripping formations can have a height extending outward from the surface of the insertion member. In certain embodiments, the gripping formations can have a height greater than about 0.1 mm, such as, e.g., a height from about 0.1 mm to about 0.8 mm, such as, e.g., a height from about 0.1 mm to about 0.6 mm, such as, e.g., a height from about 0.15 mm to about 0.5 mm, such as, e.g., a height from about 0.2 mm to about 0.4 mm, such as, e.g., a height from about 0.2 mm to about 0.3 mm, or any other suitable height.

The gripping formations can be provided on the gripping portion in any suitable location. In certain embodiments, the gripping formations can form a design, such as, e.g., a pattern. The gripping formations can be arranged to form a perceived pattern, such as, e.g., a line, a broken line, a ring, and/or a broken ring. For example, in certain embodiments, a plurality of gripping formations can be arranged to form a series of rings, such as, e.g., broken rings, by positioning two or more gripping formations at a same distance along the longitudinal axis of the applicator from the insertion end. Thus, in certain embodiments, the gripping portion can comprise multiple discontinuous rings in the axial direction, with each discontinuous ring being made up of multiple gripping formations circumferentially around the gripping portion. The rings can be provided with any suitable spacing, such as, e.g., randomly spaced, closely spaced, and/or regularly spaced from one another, both in the longitudinal direction and in the direction around the perimeter of the gripping portion.

As shown herein, in certain embodiments, the gripping formations can include elongated ridges, such as, e.g., ridges having a length and a width, with the length being greater than the width. In certain embodiments, a ridge can have a length that can be perpendicular to the longitudinal axis of the applicator. A ridge can have any suitable length, such as, e.g., from about 1 mm to about 40 mm, such as, e.g., from about 2 mm to about 30 mm, such as, e.g., from about 3 mm to about 20 mm, such as, e.g., from about 4 mm to about 15 mm, such as, e.g., from about 5 mm to about 10 mm, or any other suitable length. The ridges can extend around less than substantially the entire perimeter, such as, e.g., less than substantially the entire circumference, of the gripping portion, such as, e.g., less than about 80%, less than about 70%, less than about 60%, less than about 50%, less than about 40%, less than about 30%, less than about 20%, and/or less than about 10% of the perimeter of the gripping portion.

Ridges can be provided on the gripping portion in any suitable location. In certain embodiments, ridges can be arranged on the gripping formation to form rings, such as, e.g., discontinuous rings. Any suitable number of ridges can be arranged to form a discontinuous ring, such as, e.g., two, three, four, five, six, or more ridges. Alternatively, or in addition, ridges of the same or different sizes can be arranged along the gripping portion in the axial direction, such as, e.g., in a ladder-like formation. In certain embodiments, the perception of a ridge can be created by positioning a plurality of gripping formations, such as, e.g., dots, slashes, lines, etc., in proximity such that the perception of a ridge is created, for example, by positioning gripping formations to provide a discontinuous ridge.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An applicator comprising:
an insertion member having an insertion end and a gripping portion opposite the insertion end, the gripping portion having a circumference, the gripping portion having at least one substantially continuous ring disposed along substantially the entire circumference of the gripping portion and two or more elongated ridges having a width and a length, the length extending generally perpendicular to a longitudinal axis of the applicator, the length being greater than the width, the two or more ridges extending along less than substantially the entire circumference of the gripping portion.

2. The applicator of claim 1, wherein the gripping portion comprises three or more ridges.

3. The applicator of claim 1, wherein the at least two ridges and at least one substantially continuous ring are disposed at different distances from the insertion end along the longitudinal axis of the applicator.

4. The applicator of claim 1, wherein at least two ridges are disposed at a first and second distance from the insertion end along the longitudinal axis of the applicator and at least one substantially continuous ring is disposed at a third distance from the insertion end along the longitudinal axis of the applicator, and wherein additional ridges are disposed at the first distance and/or the second distance.

5. The applicator of claim 1, wherein the applicator is formed of paper, paperboard, cardboard, cellulose, or combinations thereof.

6. An applicator comprising:
an insertion member having an insertion end and a gripping portion opposite the insertion end, the gripping portion having a circumference, the gripping portion having a first substantially continuous ring disposed along substantially the entire circumference of the gripping portion at a first distance from the insertion end along a longitudinal axis of the applicator, a first elongated ridge extending along at least a portion of the circumference at a second distance from the insertion end along the longitudinal axis of the applicator, and a second elongated ridge extending along at least a portion of the circumference at a third distance from the insertion end along the longitudinal axis of the applicator, wherein the first ridge and the second ridge have a width and a length, the length extending generally perpendicular to a longitudinal axis of the applicator, the length being greater than the width.

7. The applicator of claim 6, wherein the first distance is greater than the second distance and the second distance is greater than the third distance.

8. The applicator of claim 6, wherein the second distance is greater than the first distance, and the first distance is greater than the third distance.

9. The applicator of claim 6, wherein the first distance is less than the second and third distances.

10. The applicator of claim 6, wherein the gripping portion further comprises a third elongated ridge extending along at least a portion of the circumference provided at a fourth distance from the insertion end along the longitudinal axis of the applicator

11. The applicator of claim 6, wherein the gripping portion further comprises a second substantially continuous ring disposed along substantially the entire circumference of the gripping portion at a fifth distance from the insertion end along a longitudinal axis of the applicator.

12. The applicator of claim 6, wherein the ridges are configured to provide a finger rest.

13. The applicator of claim 6, further comprising a one or more additional elongated ridges at the second distance, the third distance, and/or the fourth distance.

14. The applicator of claim 6, wherein the applicator is formed of paper, paperboard, cardboard, cellulose, or combinations thereof.

15. An applicator comprising:
an insertion member having an insertion end and a gripping portion opposite the insertion end, the gripping portion having a circumference, the gripping portion having at least one substantially continuous ring disposed along substantially the entire circumference of the gripping portion and two or more elongated ridges extending along less than substantially the entire circumference of the gripping portion, and
a plunger, the plunger being telescopically and slidably mounted in the insertion member, the plunger having a first end adapted for insertion into the insertion member and a second end opposite the first end, the first end having at least one raised portion adapted to interact with the at least one substantially continuous ring to retain the first end of the plunger within the insertion member absent external mechanical force.

16. The applicator of claim 15, wherein the gripping portion comprises two substantially continuous rings disposed along substantially the entire circumference of the gripping portion.

17. The applicator of claim 15, wherein the gripping portion comprises three or more ridges.

18. The applicator of claim 15, wherein the at least two ridges and at least one substantially continuous ring are disposed at different distances from the insertion end along the longitudinal axis of the applicator.

19. The applicator of claim 15, wherein at least two ridges are disposed at a first and second distance from the insertion end along the longitudinal axis of the applicator and at least one substantially continuous ring is disposed at a third distance from the insertion end along the longitudinal axis of the applicator, and wherein additional ridges are disposed at the first distance and/or the second distance.

20. The applicator of claim 15, wherein the applicator is formed of paper, paperboard, cardboard, cellulose, or combinations thereof.
